(19) United States
(12) Patent Application Publication
(10) Pub. No.: US 2012/0204895 A1
(43) Pub. Date: Aug. 16, 2012

(54) HAIR TREATMENT AGENTS COMPRISING POLYQUATERNIUM-72

(75) Inventors: Marcus Krueger, Ellerhoop (DE); Jens Delowsky, Norderstedt (DE)

(73) Assignee: Henkel AG & Co. KGaA, Dusseldorf (DE)

(21) Appl. No.: 13/455,895

(22) Filed: Apr. 25, 2012

Related U.S. Application Data
(63) Continuation of application No. PCT/EP2010/065423, filed on Oct. 14, 2010.

(30) Foreign Application Priority Data
Oct. 26, 2009 (DE) 10 2009 045 997.9

(51) Int. Cl. A61K 8/30; A61Q 5/00

(52) U.S. Cl. 132/202; 424/70.28

(57) ABSTRACT

Hair treatment agents include polyquaternium-72 and at least one amine and/or cationized amine, in particular an amidoamine and/or a cationized amidoamine.

### CROSS-REFERENCES TO RELATED APPLICATIONS

This application is a continuation of PCT/EP2010/065423, filed on Oct. 14, 2010, which claims priority under 35 U.S.C. §119 to DE 10 2009 045 997.9 filed on Oct. 26, 2009, both of which are hereby incorporated by reference.

### FIELD OF THE INVENTION

The present invention generally relates to hair treatment agents comprising at least one amine and/or cationized amine as well as the use of these agents for treating hair.

### BACKGROUND OF THE INVENTION

The importance of hair care products having the longest-lasting effect has been growing, not least of all due to the extreme stresses on hair, for example, due to dyeing and permanent waving as well as due to shampooing of hair and also environmental stresses.

However, the known active ingredients cannot adequately cover all needs. Therefore there continues to be a need for active ingredients and/or combinations of active ingredients for cosmetic agents having good care properties and good biodegradability. There is a demand for additional active care ingredients that can be incorporated with no problem into known formulations, in particular in formulations containing dyes and/or electrolytes.

Quaternary ammonium compounds of the mono-, di- and/or trialkylammonium compound type have long been known. One disadvantage of these compounds, however, is their inadequate biodegradability. Therefore, cationic compounds containing at least one ester group, the so-called ester quats have been developed. However, these exhibit a sensation which is bluntly evaluated as unpleasant and is also perceived audibly as a “squeaking” sound with respect to the feel and hold of wet hair and wet skin as well as the feel and touch of skin dried after being washed. It is therefore desirable to significantly improve upon the properties of keratinic fibers with respect to their elasticity, combability, luster and bounce of the entire hair style.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair cosmetic agent for treatment of keratinic fibers, containing at least 0.01 wt % polyquaternium-72, and at least 0.01 wt % of an amine and/or a cationized amine, in particular an amidoamine and/or a cationized amidoamine, and a cosmetic vehicle.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first embodiment of the present invention includes a composition for treating keratinic fibers, comprising
a) at least 0.01 wt % polyquaternium-72;
b) at least 0.01 wt % of an amine and/or cationized amine in particular an amidoamine and/or a cationized amidoamine; and
c) a cosmetic vehicle.

Ingredients a) and b) are described in detail below. When speaking below of an active ingredient complex (A), this statement refers to the ingredients a) and b) obligatorily contained in the agents according to the invention.

Hair treatment agents in the sense of the present invention include, for example, hair dye agents, hair bleaching agents, hair shampoos, hair conditioners, conditioning shampoos, hair sprays, hair rinses, deep conditioners for hair, hair packs, hair tonics, permanent wave fixative solutions, hair coloring shampoos, hair coloring agents, hair-setting agents, hair-waving agents, hair-styling preparations, blow-drying lotions, foam hair-setting agents, hair gels, hair waxes or combinations thereof.

Combability is understood according to the invention to refer to the combability of the wet fibers as well as the combability of the dry fibers. The measure of combability used is the combing work expended or the force expended during the operation of combing a fiber population. The skilled person may make a sensory evaluation of the measured parameters may be quantified by measurement equipment.

The feel is defined as the tactility of a fiber population, wherein the skilled person senses and evaluates the parameters of fullness and suppleness of the collective.

Shaping is understood to refer to the ability to impart a change in shape to a population of previously treated keratin-containing fibers, in particular human hair. The term “stylability” is also used in hair cosmetics.

“Bounce” is understood to refer to the vitality of a fiber composite or individual fibers, as expressed in bounce force of the moving fibers and/or of the fiber composite or the hair style as a whole.

An oxidative treatment of hair is defined according to the invention as the action of an oxidative cosmetic agent containing at least one oxidizing agent in a cosmetic vehicle.

The term “keratinic fiber” is understood to refer to animal fibers such as, for example, the hair of dogs, cats, horses, etc. as well as hoof and nails from the dog, cat or horse, for example, as well as the feathers of birds, for example, chickens. However, the term “keratinic fiber” in particular is understood to refer to human hair.

According to the invention, O/W, W/O and W/O/W emulsions in the form of creams or gels or even foaming solutions containing surfactants such as shampoos, foam aerosols or other preparations that are suitable in particular for use on hair are especially suitable as the cosmetic vehicles. However, it is also conceivable to integrate the ingredients into a powdered or even tableted formulation, which is dissolved in water before use. The cosmetic vehicles may in particular be aqueous or aqueous alcoholic.

An aqueous cosmetic vehicle contains at least 50 wt % water.

Aqueous alcoholic cosmetic vehicles in the sense of the present invention are understood to be aqueous solutions containing 3 to 70 wt % of a C₁-C₆ alcohol, in particular methanol, ethanol and/or propanol, isopropanol, butanol, isobutanol, tert-butanol, n-pentanol, isopentanol, n-hexanol, isohexanol, glycol, glycerol, 1,2-pentanediol, 1,5-pentanediol, 1,2-hexanediol or 1,6-hexanediol. The agents according to the invention may additional contain other organic solvents, for example, methoxybutanol, benzyl alcohol, ethyl diglycol or 1,2-propylene glycol. All water-soluble organic solvents are preferred.

Polyquaternium-72 is used as ingredient a). Polyquaternium-72 is hydroxyethyl cellulose that has been quaternized. Polyquaternium-72 is trimonium and cocodimonium hydroxyethyl cellulose. Polyquaternium-72 may be used either in solid form or already dissolved in an aqueous solution. Use of the commercial product Mirustyle® CP from the company Croda is preferred. Polyquaternium-72 is present in the compositions according to the invention in an amount of at least 0.01 wt %. Amounts of 0.05 wt % and most preferably amounts of at least 0.1 wt % are preferred.

In contrast with the cationic cellulose derivatives of the prior art such as in particular polyquaternium-10, the compositions according to the invention are clearly superior in particular in the bounce, the bounce behavior and the supple, velvety feel of the keratinic fibers.

The ingredient b) is at least one amine and/or cationized amine, in particular an amidoamine and/or a cationized amidoamine having one of the following structural formulas:

[R¹—NH—(CH₂)ₙ—NR²R³] (Tkat7)

and/or

[R¹—NH—(CH₂)ₙ—NR²R³R⁴]X⁻ (Tkat8)

wherein R¹ may be an acyl or alkyl radical with 6 to 30 carbon atoms, which may be branched or unbranched, saturated or unsaturated, and wherein the acyl radical and/or the alkyl radical may contain at least one OH group, and
R², R³ and R⁴ each, independently of one another, denote hydrogen or an alkyl radical with 1 to 4 carbon atoms, which may be the same or different, saturated or unsaturated, and
X⁻ is an anion, and
n is an integer between 1 and 10.

A composition in which the amine and/or the quaternized amine according to general formulas (Tkat7) and/or (Tkat8) is an amidoamine and/or a quaternized amidoamine, in which R¹ denotes a branched or unbranched, saturated or unsaturated acyl radical with 6 to 30 carbon atoms, optionally containing at least one OH group, is preferred. A fatty acid radical of oils and waxes, in particular of natural oils and waxes, is preferred here. Examples include lanolin, beeswax or candelilla wax.

Amidoamines and/or quaternized amidoamines in which R², R³ and/or R⁴ in formulas (Tkat7) and/or (Tkat8) denote a radical according to the general formula CH₂CH₂OR5 are also preferred, wherein R5 may have the meaning of alkyl radicals with 1 to 4 carbon atoms, hydroxyethyl or hydrogen. The proposed size of n in the general formulas (Tkat7) and/or (Tkat8) is an integer between 2 and 5.

Also preferred are amidoamines and/or quaternized amidoamines of the general formulas (Tkat7) and/or (Tkat8), in which the anion X⁻ is a halide ion or a compound of the genera formula RSO₃⁻, in which R has the meaning of saturated or unsaturated alkyl radicals with 1 to 4 carbon atoms.

The alkyl radical with 1 to 4 carbon atoms of R², R³ and R⁴ and/or the alkyl radical with 1 to 4 carbon atoms of RSO₃⁻ in the general formulas (Tkat7) and/or (Tkat8) may contain at least one hydroxyl group.

The alkylamidoamines may be present as such and be converted by protonation in suitable acidic solution into a quaternary compound of the composition. The cationic alkylamidoamines are preferred according to the invention.

Amidoamines, which are to be used according to the invention and may optionally be quaternized, include as amidoamines, for example: Witcamine 100 (Witco, INCI designation: cocamidopropyl dimethylamine), Incromine BB (Croda, INCI designation: behenamidopropyl dimethylamine), Mackine 401 (McIntyre, INCI designation: isostearylamidopropyl dimethylamine) and other Mackine products, Adogen S18V (Witco, INCI designation: stearylamidopropyl dimethylamine) and as permanently cationic amidoamines: Rewoquat RTM 50 (Witco Surfactants GmbH, INCI designation: ricinoleamidopropyltrimonium methosulfate), Empigen CSC (Albright & Wilson, INCI designation: cocamidopropyltrimonium chloride), Swanol Lanoquat DES-50 (Nikko, INCI designation: quaternium-33), Rewoquat UTM 50 (Witco Surfactants GmbH, undecyleneamidopropyltrimonium methosulfate). Stearyl amidopropyldimethylamine is preferred as the amidoamine.

The ingredients b) according to the invention are used in amounts of at least 0.01 wt %. Amounts of 0.01 to 10.0 wt %, especially preferably from 0.01 to 7.5 wt %, most preferably from 0.05 to 5.0 wt % and in particular from 0.1 to 5.0 wt % are preferred.

Ingredients a) and b) are preferably used in a weight ratio of 2:1 to 1:5. A ratio 2:1 to 1:3 and most preferably from 1:1 to 1:2 is preferred.

Furthermore, it may be preferable if in addition to the active ingredients a) and b) according to the invention, no other polymers and/or no other cationic surfactants are present in the compositions according to the invention.

In addition, it has been found that the effect of the active ingredient complex described above can be substantially increased further if a vitamin or a vitamin precursor of the vitamin B complex is additionally used.

The vitamin B group or the vitamin B complex includes, among other things:
Vitamin B₁ (thiamine)
Vitamin B₂ (riboflavin)
Vitamin B₃. This designation often includes the compounds nicotinic acid and nicotinamide (niacin amide). According to the invention nicotinamide is preferred and is present in the agents according to the invention preferably in amounts of 0.05 to 1 wt %, based on the total agent.
Vitamin B₅ (pantothenic acid, panthenol and pantolactone). Of this group, panthenol and/or pantolactone are preferred for use here. Derivatives of panthenol that may be used according to the invention include in particular the esters and ethers of panthenol as well as cationically derivatized panthenols. Individual representatives include, for example, panthenol triacetate, panthenol monoethyl ether and its monoacetate as well as the cationic panthenol derivatives. The afore mentioned compounds of the vitamin B₅ type are preferably present in the agents according to the invention in amounts of 0.05-10 wt %, based on the total agent. Amounts of 0.1-5 wt % are especially preferred.
Vitamin B₆ (pyridoxine and pyridoxamine and pyridoxal).

Of the vitamin B complex, vitamins of the B₃ complex and B₅ complex are preferred in particular.

Additional ingredients that are used are surfactants, wherein both anionic and zwitterionic, ampholytic, nonionic and cationic surfactants are suitable in principle. The choice depends on the type of agent. In particular at least one surfactant from the group of anionic, zwitterionic or nonionic surfactant substances is selected in the case of a shampoo. It is preferable here for at least one anionic and at least one zwitterionic surfactant surface-active substance to be selected. These surface-active substances are especially preferably selected from the group of especially mild surface-active substances, where the ratio between anionic and zwitterionic surface-active substances is between 10:1 and 1:5. The ratio is especially preferably 5:1 to 1:2.

Suitable anionic surfactants (Tanion) in the preparations according to the invention include all the anionic surface-active substances that are suitable for use on the human body. Typical examples of anionic surfactants include:
linear and branched fatty acids with 8 to 30 carbon atoms (soaps),
ether carboxylic acids of the formula R—O—(CH₂CH₂O)ₓ—CH₂—COOH, in which R is a linear alkyl group with 8 to 30 carbon atoms and x=0 or 1 to 16, and their salts,
acyl sarcosides with 8 to 24 carbon atoms in the acyl group,
acyl taurides with 8 to 24 carbon atoms in the acyl group,
acyl isethionates with 8 to 24 carbon atoms in the acyl group,
sulfosuccinic acid mono- and dialkyl esters with 8 to 24 carbon atoms in the alkyl group and sulfosuccinic acid monoalkylpolyoxyethyl esters with 8 to 24 carbon atoms in the alkyl group and 1 to 6 oxyethyl groups,
linear alkane sulfonates with 8 to 24 carbon atoms,
linear α-olefin sulfonates with 8 to 24 carbon atoms,
α-sulfo fatty acid ethyl esters of fatty acids with 8 to 30 carbon atoms,
alkyl sulfates and alkyl polyglycol ether sulfates of the formula R—O(CH₂CH₂O)ₓ—OSO₃H in which R denotes a preferably linear alkyl group with 8 to 30 carbon atoms and x is 0 or 1 to 12,
hydroxysulfonates corresponding essentially to at least one of the two following formulas or their mixtures as well as their salts: CH₃—(CH₂)_{y}—CHOH—(CH₂)ₚ—(CH—SO₃M)-(CH₂)_{z}—CH₂—O—(CₙH₂ₙO)ₓ—H and/or CH₃—(CH₂)_{y}—(CH—SO₃M)-(CH₂)ₚ—CHOH—(CH₂)_{z}—CH₂—O—(CₙH₂ₙO)ₓ—H wherein y and z=0 in both formulas or integers from 1 to 18, p=0, 1 or 2 and the sum (y+z+p) is a number from 12 to 18, x=0 or a number from 1 to 30 and n is an integer from 2 to 4 and M=H or alkali, in particular sodium, potassium, lithium, alkaline earth, in particular magnesium, calcium, zinc and/or an ammonium ion which may optionally be substituted, in particular mono-, di-, tri- or tetraammonium ions with C₁ to C₄ alkyl, alkenyl or aryl radicals,
sulfated hydroxyalkylpolyethylene and/or hydroxyalkylene propylene glycol ether of the formula R¹—(CHOSO₃M)-CHR³—(OCHR⁴—CH₂)ₙ—OR² where R¹ stands for a linear alkyl radical with 1 to 24 carbon atoms, R² stands for a linear or branches, saturated alkyl radical with 1 to 24 carbon atoms, R³ stands for hydrogen or a liar alkyl radical with 1 to 24 carbon atoms, R⁴ stands for hydrogen or a methyl radical and M stands for hydrogen, ammonium, alkyl ammonium, alkanol ammonium, wherein the alkyl and alkanol radicals each have 1 to 4 carbon atoms, or a metal atom selected from lithium, sodium, potassium, calcium or magnesium, and n stands for a number in the range of 0 to 12 and also the total number of carbon atoms present in R¹ and R³ is 2 to 44,
sulfonates of unsaturated fatty acids with 8 to 24 carbon atoms and 1 to 6 double bonds,
esters of tartaric acid and citric acid with alcohols which are the addition products of approx. 2-15 molecules of ethylene oxide and/or propylene oxide onto fatty alcohols with 8 to 22 carbon atoms,
alkyl and/or alkenyl ether phosphates of the formula R¹(OCH₂CH₂)ₙ—O—(PO—OX)—OR² in which R¹ preferably stands for an aliphatic hydrocarbon radical with 8 to 30 carbon atoms, R² stands for hydrogen, a (CH₂CH₂O)ₙR² radical or X, n stands for numbers from 1 to 10 and X stands for hydrogen, an alkali or alkaline earth metal or NR³R⁴R⁶R⁶, where R³ to R⁶, independently of one another, stand for hydrogen or a C₁ to C₄ hydrocarbon radical,
sulfated fatty acid alkylene glycol esters of the formula RCO(AlkO)ₙSO₃M, in which RCO— stands for a linear or branched, aliphatic, saturated and/or unsaturated acyl radical with 6 to 22 carbon atoms, Alk stands for CH₂CH₂, CHCH₃CH₂ and/or CH₂CHCH₃, n stands for numbers from 0.5 to 5 and M stands for a metal such as alkali metal in particular sodium, potassium, lithium, alkaline earth metal in particular magnesium, calcium, zinc or ammonium ion such as ⁺NR³R⁴R⁵R⁶ where R³ to R⁶, independently of one another, stand for hydrogen or a C₁ to C₄ hydrocarbon radical,
monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)_{z}—SO₃X, in which R⁸CO stands for a linear or branched, acyl radical with 6 to 22 carbon atoms, x, y and z amount to a total of 0 or stand for numbers from 1 to 30, preferably 2 to 10, and X stands for an alkali or alkaline earth metal. Typical examples of monoglyceride (ether) sulfates suitable in the sense of the invention include the reaction products of lauric acid monoglyceride, coconut fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulfonic acid in the form of the sodium salts. Monoglyceride sulfates, in which R⁸CO stands for a linear acyl radical with 8 to 18 carbon atoms, are preferred,
amide ether carboxylic acids, R¹—CO—NR²—CH₂CH₂—O—(CH₂CH₂O)ₙ—CH₂COOM, in which R¹ is a linear or branched alkyl or alkenyl radical with 2 to 30 carbon atoms in the chain, n stands for an integer from 1 to 20, and R² stands for hydrogen, a methyl, ethyl, propyl, isopropyl, n-butyl, tert-butyl or isobutyl radical, and M stands for hydrogen or a metal, such as an alkali metal, in particular sodium, potassium, lithium, alkaline earth metal, in particular magnesium, calcium, zinc or an ammonium ion, such as ⁺NR³R⁴R⁵R⁶, in which R³ to R⁶, independently of one another, stand for hydrogen or a C₁ to C₄ hydrocarbon radical. Such products are available under the brand name Akypo® from the company Chem-Y, for example,
acyl glutamates of the formula XOOC—CH₂CH₂CH(C(NH)OR)—COOX in which RCO stands for a linear or branched acyl radical with 6 to 22 carbon atoms and 0 and/or 1, 2 or 3 double bonds and X stands for hydrogen, an alkali and/or alkaline earth metal, ammonium, alkyl ammonium, alkanol ammonium or glucammonium
condensation products of a water-soluble salt of a water-soluble protein hydrolyzate with a C₈ to C₃₀ fatty acid. Such products have long been available commercially under the brand names Lamepon®, Maypon®, Gluadin®, Hostapon® KCG or Amisoft®.
alkyl and/or alkenyl oligoglycoside carboxylates, sulfates, phosphates and/or isethionates,
acyl lactylates [lactates] and
hydroxy mixed ether sulfates.

If the mild anionic surfactants contain polyglycol ether chains, it is most especially preferred that they have a narrow homolog distribution. In addition, in the case of mild anionic surfactants with polyglycol ether units it is preferable for the number of glycol ether groups to be 1 to 20, preferably 2 to 15, especially preferably 2 to 12. Especially mild anionic surfactants with polyglycol ether groups without a narrow homolog distribution may also be obtained, for example, if on the one hand the number of polyglycol ether groups is 4 to 12 and Zn or Mg ions are selected as the counterion. One example of this is the commercial product Texapon® ASV.

Especially zwitterionic surfactants include the so-called betaines such as the N-alkyl-N,N-dimethylammonium glycinates, for example, the coconut alkyl dimethylammonium glycinate, N-acylaminopropyl-N,N-dimethylammonium glycinates, for example, the coconut acylaminopropyldimethylammonium glycinate and 2-alkyl-3-carboxymethyl-3-hydroxyethylimidazolines, each with 8 to 18 carbon atoms in the alkyl or acyl group as well as coconut acylaminoethylhydroxyethylcarboxymethyl glycinate. A preferred zwitterionic surfactant is the fatty acid amide derivative known by the INCI designation cocamidopropyl betaine.

Ampholytic surfactants (Tampho) are understood to be surface-active compounds capable of forming internal salts Examples of suitable ampholytic surfactants include N-alkylglycine, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-dipropionic acids and alkylaminoacetic acids each with approx. 8 to 24 carbon atoms in the alkyl group. Typical examples of amphoteric and/or zwitterionic surfactants include alkylbetaines, alkylamidobetaines, aminopropionates, aminoglycinates, imidazolinium betaines and sulfobetaines.

Especially preferred ampholytic surfactants include N-cocoalkylamino-propionate, cocoacyaminoethylaminopropionate and C₁₂-C₁₈ acylsarcosine.

Nonionic surfactants (Tnio) include for example
addition products of 2 to 50 mol ethylene oxide and/or 0 to 5 mol propylene oxide onto linear and branched fatty alcohols with 6 to 30 carbon atoms, fatty acid polyglycol ether and/or fatty alcohol polypropylene glycol ethers and/or mixed fatty alcohol polyethers,
addition products of 2 to 50 mol ethylene oxide and/or 0 to 5 mol propylene oxide onto linear and branched fatty alcohols with 6 to 30 carbon atoms, fatty acid polyglycol ether and/or fatty alcohol polypropylene glycol ethers and/or mixed fatty alcohol polyethers,
addition products of 2 to 50 mol ethylene oxide and/or 0 to 5 mol propylene oxide onto linear and branched alkyl phenols with 8 to 15 carbon atoms in the alkyl group, alkyl phenol polyglycol ethers and/or alkyl polypropylene glycol ethers and/or mixed alkyl phenol polyethers,
end-group-capped addition products of 2 to 50 mol ethylene oxide and/or 0 to 5 mol propylene oxide onto linear and branched fatty alcohols with 8 to 30 carbon atoms, onto fatty acids with 8 to 30 carbon atoms and onto alkyl phenols with 8 to 15 carbon atoms in the alkyl group, with end group capping with a methyl group or a C₂ to C₆ alkyl group, such as those products obtainable under the brand names Dehydrol® LS, Dehydrol® LT (Cognis), for example,
C₁₂-C₃₀ fatty acid mono- and diesters of addition products of 1 to 30 mol ethylene oxide onto glycerol
addition products of 5 to 60 mol ethylene oxide onto castor oil and hydrogenated castor oil,
polyol fatty acid esters, for example, the commercial product Hydagen® HSP (Cognis) or Sovermol products (Cognis),
alkoxylated triglycerides,
alkoxylated fatty acid alkyl esters of the formula (Tnio-1) R¹CO—(OCH₂CHR²)_{w}OR³ (Tnio-1)
in which R¹CO stands for a linear or branched saturated and/or unsaturated acyl radical with 6 to 22 carbon atoms, R² stands for hydrogen or methyl, R³ stands for linear or branched alkyl radicals with 1 to 4 carbon atoms and w stands for numbers from 1 to 20,
amine oxides,
hydroxy mixed ethers R¹O[CH₂CH(CH₃)O]ₓ(CH₂CHR²O)_{y}[CH₂CH(OH)R³]_{Z} where R¹ stands for a linear or branched, saturated or unsaturated alkyl and/or alkenyl radical with 2 to 30 carbon atoms, R² stands for hydrogen, a methyl, ethyl, propyl or isopropyl radical, R³ stands for a linear or branched alkyl radical with 2 to 30 carbon atoms, x stands for 0 or a number from 1 to 20, Y stands for a number from 1 to 30 and z stands for the number 1, 2, 3, 4 or 5.
sorbitan fatty acid esters and addition products of ethylene oxide onto sorbitan fatty acid esters such as polysorbates,
sugar fatty acid esters and addition products of ethylene oxide onto sugar fatty acid esters,
addition products of ethylene oxide onto fatty acid alkanolamides and fatty amines,
sugar surfactants of the type of alkyl and alkenyl oligoglycosides
sugar surfactants of the type of fatty acid N-alkylpolyhydroxyalkylamides,
fatty acid amide polyglycol ethers, fatty amine polyglycol ethers,
mixed ethers and/or mixed formals and polysorbates.

In addition, a quaternary imidazoline compound, i.e., a compound having a positively charged imidazoline ring is also preferred as the cationic surfactant. Formula I shown below gives the structure of these compounds

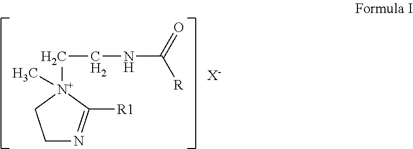

The radicals R and R1, independently of one another, each stand for a saturated or unsaturated, linear or branched hydrocarbon radical with a chain length of 8 to 30 carbon atoms. The preferred compounds of formula I each contain the same hydrocarbon radical for R and R1. The chain length of the radicals R and R1 is preferably 12 carbon atoms. Compounds with a chain length of at least 16 carbon atoms and most especially preferably with at least 20 carbon atoms are especially preferred. An extremely preferred compound of formula I has a chain length of 21 carbon atoms. A product of this chain length is available, for example, under the brand name quatemium-91 or the commercial designations Crodazosoft® DBQ which contains, in addition to quatemium-91, also cetrimonium methosulfate and cetearyl alcohol as well as Crodazosoft® SCQ, which also contains PPG-3 benzyl ether myristate in addition to quaternium-91. Special examples according to the invention can be obtained, for example, under the INCI designations quaternium-27, quaternium-72, quaternium-83 and quatemium-91. The commercial products Crodazosoft® DBQ and Crodazosoft® SCQ and/or quaternium-91 are the most preferred.

The imidazolines of formula I are present in the compositions according to the invention in amounts of 0.01 to 20 wt %, preferably in amounts of 0.05 to 10 wt % and most especially preferably in amounts of 0.1 to 7.5 wt %. The best results are obtained with quantities of 0.1 to 5 wt %, each based on the total composition of the respective agent.

In addition, the following cationic surfactants according to the formula (Tkat-2) may be used

RCO—X—N⁺R¹R²R³A⁻ (Tkat-2)

R here stands for a substituted or unsubstituted, branched or linear alkyl or alkenyl radical with 11 to 35 carbon atoms in the chain
X stands for O or NR⁵,
R¹ stands for an alkylene group with 2 to 6 carbon atoms which may be substituted or unsubstituted such that in the event of a substitution, the substitution with an OH group or an NH group is preferred,
R², R³ each independently of the others represents an alkyl or hydroxyalkyl group with 1 to 6 carbon atoms in the chain, where the chain may be linear or branched.

R⁵ stands for hydrogen or a C₁ to C₆ linear or branched alkyl or alkenyl radical which may also be substituted by a hydroxyl group.

Within this structural class, the compounds of one of the following structures are preferably used:

CH₃(CH₂)₂₀CONH(CH₂)₃—N⁺(CH₃)₂—CH₂CH₃ A (Tkat-3)

CH₃(CH₂)₂₀CONH(CH₂)₃—N⁺(CH₃)₂—CH₂(CHOH)CH₂OH A (Tkat-4)

CH₃(CH₂)₂₀COOCHCHOHCH₂—N⁺(CH₃)₃ A (Tkat-5)

CH₃(CH₂)₂₀CONH(CH₂)₃—N⁺(CH₃)₂—CH₂CH₂OH A (Tkat-6)

Examples of such commercial products include Schercoquat BAS, Lexquat AMG-BEO, Akypoquat 131 or Incroquat Behenyl HE.

Furthermore, ester quats according to the formula (Tkat1-2) may also be used

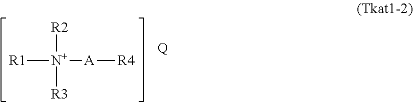

in which the radicals R1, R2 and R3 are each independent of one another and may be the same or different. The radicals R1, R2 and R3 denote:
a branched or unbranched alkyl radical with 1 to 4 carbon atoms which may contain at least one hydroxyl group or
a saturated or unsaturated branched or unbranched or a cyclic saturated or unsaturated alkyl radical with 6 to 30 carbon atoms which may contain at least one hydroxyl group or
an aryl or alkaryl radical, for example, phenyl or benzyl
the radical (A-R4) with the provision that at most two of the radicals R1, R2 or R3 may stand for this radical.

The radical (A-R4) is present at least one to three times.

In this formula, A stands for:
1) (CH₂)ₙ where n=1 to 20 preferably n=1 to 10 and especially preferably n=1-5 or
2) (CH₂—CHR5-O)ₙ where n=1 to 200, preferably 1 to 100, especially preferably 1 to 50 and especially preferably 1 to 20 with R5 being present in the meaning of hydrogen, methyl or ethyl,
   and R4 stands for:
1) R6-O—CO, wherein R6 is a saturated or unsaturated, branched or unbranched or cyclic saturated or unsaturated alkyl radical with 6 to 30 carbon atoms, which may contain at least one hydroxyl group and which may optionally also be ethoxylated with 1 to 100 ethylene oxide units and/or 1 to 100 propylene oxide units, or
2) R7-CO, wherein R7 denotes a saturated or unsaturated, branched or unbranched or cyclic saturated or unsaturated alkyl radical with 6 to 30 carbon atoms, which may contain at least one hydroxyl group and which may also be ethoxylated with 1 to 100 ethylene oxide units and/or 1 to 100 propylene oxide units,
   and Q stands for a physiologically tolerable organic or inorganic anion.

Such products are distributed, for example, under the brand names Rewoquat®, Stepantex®, Dehyquart® and Armocare®. The products Armocare® VGH-70, an N,N-bis(2-palmitoyloxyethyl)dimethylammonium chloride and Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such ester quats.

Additional compounds of the formula (Tkat1-2) which are especially preferred according to the invention fall under the formula (Tkat1-2.1), the cationic betaine esters.

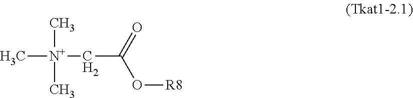

R8 corresponds in its meaning to R7.

As an additional ingredient, monoalkyltrimethylammonium salts with a chain length of the alkyl radical of 16 to 24 carbon atoms may also be present.

These compounds have the structure represented in the formula (Tkat1-1):

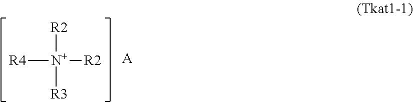

wherein R1, R2 and R3 each stand for a methyl group and R4 stands for a saturated, branched or unbranched alkyl radical with a chain length of 16 to 24 carbon atoms.

Examples of compounds of the formula (Tkat1-1) include cetyl trimethylammonium chloride, cetyl trimethylammonium bromide, cetyl trimethylammonium methosulfate, stearyl trimethylammonium chloride, behenyl trimethylammonium chloride, behenyl trimethylammonium bromide and behenyl trimethylammonium methosulfate.

The anion of all the cationic structural formulas given above for all the cationic compounds listed above is selected from the physiologically tolerable anions. Examples include the halide ions, fluoride, chloride, bromide, sulfate of the general formula RSO₃⁻ in which R has the meaning of saturated or unsaturated alkyl radicals with 1 to 4 carbon atoms, or anionic radicals of organic acids such as maleate, fumarate, oxalate, tartrate, citrate, lactate or acetate.

The cationic surfactants mentioned above may be used individually or in any combinations with one another, amounts between 0.01 and 20 wt %, preferably amounts of 0.01 to 10 wt % and most especially preferably amounts of 0.1 to 7.5 wt % being present. The best results are obtained with amounts of 0.1 to 5 wt %, each based on the total composition of the respective agent.

The surfactants (T) are used in amounts of 0.05-45 wt %, preferably 0.1 30 wt % and most especially preferably 0.5-25 wt %, based on the total agent used according to the invention.

The most highly preferred compositions comprising the active ingredient combination (A) according to the invention are characterized in that they additionally contain at least one native oil or at least one ester oil or at least one silicone oil.

Ester oils are understood to refer to the esters of C₆-C₃₀ fatty acids with C₂-C₃₀ fatty alcohols. The monoesters of fatty acids with alcohols with 2 to 24 carbon atoms are preferred. Examples of fatty acid components that may be used in the esters include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucaic acid as well as their technical-grade mixtures. Examples of the fatty alcohol fractions in the ester oils include isopropyl alcohol, capric alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl, alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol as well as their technical-grade mixtures. Especially preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut alcohol—caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

The ester oils may of course also be alkoxylated with ethylene oxide, propylene oxide or mixtures of ethylene oxide and propylene oxide. The alkoxylation may be located on the fatty alcohol part as well as on the fatty acid part or even on both parts of the ester oils. However, it is preferable according to the invention if the fatty alcohol has been alkoxylated first and then esterified with the fatty acid. These compounds are represented in general in the formula (D4-II)

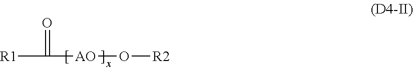

where R1 stands for a saturated or unsaturated, branched or unbranched, cyclic saturated [or]cyclic unsaturated acyl radical with 6 to 30 carbon atoms,
AO stands fro ethylene oxide, propylene oxide or butylene oxide,
x stands for a number between 1 and 200, preferably 1 and 100, especially preferably between 1 and 50, most especially preferably between 1 and 20, most preferably between 1 and 10 and especially most preferably between 1 and 5,
R2 stands for a saturated or unsaturated, branched or unbranched, cyclic saturated or cyclic unsaturated alkyl, alkenyl, alkynyl, phenyl or benzyl radical with 6 to 30 carbon atoms. Examples of fatty acid components that may be used as radical R1 in the esters include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucaic acid as well as their technical-grade mixtures. Examples of the fatty alcohol components as radical R2 in the ester oils include benzyl alcohol, isopropyl alcohol, capric alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol as well as their technical-grade mixtures. An especially preferred ester oil according to the invention is available, for example, under the INCI designation PPG-3 benzyl ether myristate.

In addition, the ester oils are understood to include:
dicarboxylic acid esters such as di-n-butyl adipate, di-(2-ethylhexyl)adipate, di-(2-ethylhexyl)succinate and diisotridecyl acelate as well as diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate, as well as
symmetrical, asymmetrical or cyclic esters of carbonate acid with fatty alcohols, for example, glycerol carbonate or dicaprylyl carbonate (Cetiol® CC),
trifatty acid esters of saturated and/or unsaturated linear and/or branched fatty acids with glycerol,
   fatty acid partial glycerides, i.e., monoglycerides, diglycerides and their technical-grade mixtures. Typical examples include mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucaic acid as well as their technical-grade mixtures. Oleic acid monoglycerides are preferred for use here.

The ester oils are used in the agents according to the invention in an amount of 0.01 to 20 wt %, preferably 0.01 to 10.0 wt %, especially preferably 0.01 to 7.5 wt %, most preferably 0.1 to 5.0 wt %. According to the invention it is of course also possible to use several ester oils at the same time.

In addition, natural oils may also be used with the active ingredient combination (A) according to the invention. The natural oils may also be waxy or solid at room temperature. These oils, oil substances, preferably have a melting point of less than 50° C., especially preferably less than 45° C., most especially preferably less than 40° C., most especially preferably less than 35° C. and most preferably the cosmetic oils are flowable at a temperature of less than 30° C. According to the invention, the natural oils are also understood to include synthetic oils such as hydrocarbons. These oils are defined and described in greater detail below. The oils according to the invention include, for example:
vegetable oils. Examples of such oils include sunflower oil, olive oil, soy oil, canola oil, almond oil, jojoba oil, orange oil, wheat germ oil, peach pit oil and the liquid fractions of coconut oil. However, other triglyceride oils such as the liquid fraction of beef tallow and synthetic triglyceride oils are also suitable;
   liquid paraffin oils, isoparaffin oils and synthetic hydrocarbons such as di-n-alkyl ethers with a total of between 12 and 36 carbon atoms, in particular 12 to 24 carbon atoms such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert-butyl-n-octyl ether, isopentyl-n-octyl ether and 2-methylpentyl-n-octyl ether. The compounds 1,3-di-(2-ethylhexyl)cyclohexane (Cetiol® S) and di-n-octyl ether (Cetiol® OE) which are available as commercial products may be preferred.

Examples of natural oils that may be considered according to the invention include amaranth seed oil, apricot kernel oil, argan oil, avocado oil, babasu oil, cottonseed oil, borage seed oil, camelina oil, thistle oil, peanut oil, pomegranate seed oil, grapefruit seed oil, hemp oil, hazel nut oil, elderberry seed oil, black current seed oil, jojoba oil, cocoa butter, linseed oil, macadamia nut oil, corn germ oil, almond oil, manila oil, evening primrose oil, olive oil, palm oil, canola oil, rice oil, sea buckthorn berry oil, sea buckthorn kernel oil, sesame oil, rhea butter, soy oil, sunflower oil, grapeseed oil, walnut oil or wild rose oil.

The natural oils are used in the agents according to the invention in an amount of 0.01 to 20 wt %, preferably 0.01 to 10.0 wt %, especially preferably 0.01 to 7.5 wt %, most preferably from 0.1 to 5.0 wt %. It is of course also possible according to the invention to use several of the natural oils at the same time.

As an additional extremely preferred ingredient to increase the effect of the active ingredient complex (A), the agents according to the invention preferably contain at least one silicone polymer selected from the group of dimethiconols and/or the group of amino-functional silicones and/or the group of dimethicones and/or the group of cyclomethicones. These ingredients are described below

The dimethicones according to the invention may be linear or branched as well as cyclic or cyclic and branched. Linear dimethicones may be represented by the following structural formula (Si1):

(SiR¹₃)—O—(SiR²₂—O)ₓ—(SiR¹₃) (Si1)

Branched dimethicones may represented by the structural formula (Si1.1):

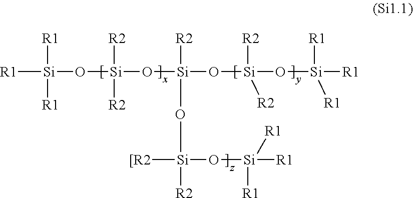

The radicals R¹ and R², independently of one another, stand for hydrogen, a methyl radical, a C₂ to C₃₀ linear, saturated or unsaturated hydrocarbon radical, a phenyl radical and/or an aryl radicals. The numbers x, y and z are integers and run, independently of one another, from 0 to 50,000. The molecular weights of the dimethicones are between 1000 D and 10,000,000 D. The viscosities are between 100 and 10,000,000 cPs measured at 25° C. with the help of a glass capillary viscometer according to the Dow Corning Corporate test method CTM 0004 of Jul. 20, 1970. Preferred viscosities are between 1000 and 5,000,000 cPs, most especially preferred viscosities are between 10,000 and 3,000,000 cPs. The most preferred range is between 50,000 and 2,000,000 cPs. Most preferred viscosities are around the range of approx. 60,000 cPs. The product Dow Corning 200 with 60,000 cSt may be mentioned here as an example.

Especially preferred cosmetic or dermatological preparations according to the invention are characterized in that they contain at least one silicone of the formula (Si1.2)

(CH₃)₃Si—[O—Si(CH₃)₂]ₓ—O—Si(CH₃)₃ (Si1.2)

in which x stands for a number from 0 to 100, preferably from 0 to 50, more preferably from 0 to 20 and in particular 0 to 10.

The dimethicones (Si1) are present in the compositions according to the invention in amounts of 0.01 to 10 wt %, preferably 0.01 to 8 wt %, especially preferably 0.1 to 7.5 wt % and in particular 0.1 to 5 wt %, based on the total composition.

Especially preferred agents according to the invention contain one or more amino-functional silicones. Such silicones may be described, for example, by the formula (Si2)

M(RₐQ_{b}SiO_{(4-a-b)/2})ₓ(R_{c}SiO_{(4-c)/2})_{y}M (Si2)

wherein in the above formula
R is a hydrocarbon or a hydrocarbon radical with 1 to approx. 6 carbon atoms,
Q is a polar radical of the general formula R¹HZ,
wherein
R¹ is a divalent bonding group which is bound to hydrogen and the Z radical, composed of carbon and hydrogen atoms, carbon, hydrogen and oxygen atoms or carbon, hydrogen and nitrogen atoms and
Z is an organic, amino-functional radical containing at least one amino-functional group;
a assumes values in the range from approx. 0 to approx. 2,
b assumes values in the range from approx. 1 to approx. 3,
a+b is less than or equal to 3 and
c is a number in the range from approx. 1 to approx. 3 and
x is a number in the range from 1 to approx. 2000, preferably from approx. 3 to approx. 50 and most preferably from approx. 3 to approx. 25 and
y is a number in the range from approx. 20 to approx. 10,000, preferably from approx. 125 to approx. 10,000 and most preferably from approx. 150 to approx. 1000, and
M is a suitable silicone end group such as those known in the prior art, preferably trimethylsiloxy.

Z according to formula (Si2) is an organic, amino-functional radical comprising at least one functional amino group. One possible formula for said Z is NH(CH₂)_{z}NH₂, wherein z is an integer greater than or equal to 1. Another possible formula for said Z is NH(CH₂)_{z}(CH₂)_{zz}NH, wherein both z and zz, independently of one another, denote an integer greater than or equal to 1, such that this structure comprises diamino ring structures such as piperazinyl. Said Z is most preferably an NHCH₂CH₂NH₂ radical. Another possible formula for said Z is N(CH₂)_{z}(CH₂)_{zz}NX₂ or NX₂ wherein each X is selected independently of X₂ and is from the group consisting of hydrogen and alkyl groups with 1 to 12 carbon atoms, and zz is 0.

Q according to formula (Si2) is most preferably a polar amino-functional radical of the formula CH₂CH₂CH₂NHCH₂CH₂NH₂.

In formula (Si2) a assumes values in the range of 0 to 2, b assumes values in the range of 2 to 3, a+b is equal to or less than 3 and c is a number in the range of 1 to 3. Cationic silicone oils such as the commercially available products Dow Corning (DC) 929 emulsion, DC 2-2078, DC 5-7113, SM-2059 (General Electric) as well as SLM-55067 (Wacker) are suitable according to the invention.

Especially preferred agents according to the invention are characterized in that they contain at least one amino-functional silicone of formula (Si3a)

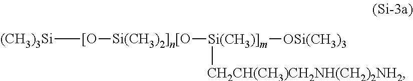

wherein m and n are numbers, whose sum (m+n) is between 1 and 2000 preferably between 50 and 150, where n preferably assumes values from 0 to 1999 and in particular from 49 to 149 and m preferably assumes values from 1 to 2000 in particular from 1 to 10.

These silicones are designated as trimethylsilyldimethicones according to the INCI declaration and are available, for example, under the brand name Q2-7224 (manufacturer Dow Corning; a stabilized trimethylsilylamodimethicone).

A gents according to the invention containing at least one amino-functional silicone of the formula (Si3b) are especially preferred

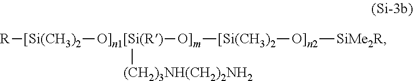

wherein
R stands for OH, a C₁ to C₂₀ alkoxy group (optionally ethoxylated and/or propoxylated) or a CH₃ group,
R′ stands for OH, a C₁ to C₂₀ alkoxy group or a CH₃ group and
m, n1 and n2 are numbers, whose sum (m+n1+n2) is between 1 and 2000, preferably between 50 and 150, where the sum (n1+n2) preferably assumes values from 0 to 1999 and in particular from 49 to 149 and m preferably assumes values from 1 to 2000 and in particular from 1 to 10.

These silicones are designated according to the INCI declaration as amodimethicones and/or as functionalized amodimethicones, for example, bis(C₁₃₋₁₅ alkoxy) PG amodimethicone (for example as the commercial product DC 8500 from the company Dow Corning), trideceth-9 PG-amodimethicone (for example available as the commercial product Silcare Silicone SEA from the company Clariant).

Suitable diquaternary silicones are selected from compounds of the general formula (Si3c)

[R¹R²R³N⁻-A-SiR⁷R⁸—(O—SiR⁹R¹⁰)ₙ—O—SiR¹¹R¹²-A-N⁺R⁴R⁵R⁶]2X⁻ (Si3c)

wherein the radicals R¹ to R⁶, independently of one another, denote C₁ to C₂₂ alkyl radicals which may contain hydroxyl groups and preferably at least one of the radicals has at least 8 carbon atoms and the other radicals have 1 to 4 carbon atoms, the radicals R⁷ to R¹², independently of one another, may be the same or different and denote C₁ to C₁₀ alkyl or phenyl, A is a divalent organic compound group,
n is a number from 0 to 200 preferably from 10 to 120 especially preferably from 10 to 40 and X⁻ is an anion.

The divalent compound group is preferably a C₁ to C₁₂ alkylene or alkoxyalkylene group which may be substituted with one or more hydroxyl groups. Especially preferred is the group (CH₂)₃—O—CH₂—CH(OH)—CH₂.

The anion X⁻ may be a halide ion, an acetate, an organic carboxylate or a compound of the general formula RSO₃⁻ wherein R has the meaning of C₁ to C₄ alkyl radicals.

A preferred diquaternary silicone has the general formula (Si3d)

[RN⁺Me₂-A-(SiMe₂O)ₙ—SiMe₂-A-N⁺Me₂R]2CH₃COO⁻ (Si3d)

wherein A is the group (CH₂)₃—O—CH₂—CH(OH)—CH₂,
R is an alkyl radical with at least 8 carbon atoms and n is a number from 10 to 120.

Suitable silicone polymers having two terminal quaternary ammonium groups are known under the INCI designation quatemium-80. These are dimethylsiloxanes with two terminal trialkylammonium groups. Such diquaternary polydimethylsiloxane are distributed by the company Evonik under the brand names Abil® Quat 3270, 3272 and 3474.

Cosmetic or dermatological preparations preferred according to the invention are characterized in that they contain, based on their weight, 0.01 to 10 wt %, preferably 0.01 to 8 wt %, especially preferably 0.1 to 7.5 wt % and in particular 0.2 to 5 wt % amino-functional silicone(s) and/or diquaternary silicone.

The compositions according to the invention may contain at least one polyammonium polysiloxane compound as the silicone. The polyammonium polysiloxane compounds may be obtained, for example, under the brand name Baysilone® from GE Bayer Silicones. The products with the designations Baysilone TP 3911, SME 253 and SFE 839 are preferred. Most especially preferred is the use of Baysilone TP 3911 as the active component of the compositions according to the invention. The polyammonium polysiloxane compounds are used in the compositions according to the invention in an amount of 0.01 to 10 wt %, preferably 0.01 to 7.5 wt %, especially preferably 0.01 to 5.0 wt %, most especially preferably from 0.05 to 2.5 wt %, each based on the total composition.

Cyclic siloxanes, which are cyclic dimethicones known according to the INCI as cyclomethicones, can be used to advantage according to the invention. Cosmetic or dermatological preparations according to the invention containing at least one silicone of the formula (Si-4)

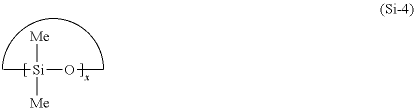

in which x stands for a number from 3 to 200 preferably from 3 to 10 more preferably from 3 to 7 and in particular 3, 4, 5 or 6 are preferred here.

According to the agents according to the invention, which are also preferred are characterized in that they contain at least one silicone of the formula (Si-5)

R³Si—[O—SiR]ₓ—(CH₂)ₙ—[O—SiR₂]_{y}—O—SiR³ (Si-5)

in which R stands for the same or different radicals from the group H, phenyl, benzyl, CH₂—CH(CH₃)Ph, C₁ to C₂₀ alkyl radicals, preferably CH₃, CH₂CH₃, CH₂CH₂CH₃, CH(CH₃)₂, CH₂CH₂CH₂H₃, CH₂CH(CH₃)₂, CH(CH₃)CH₂CH₃, C(CH₃)₃, x and/or y stand(s) for a number from 0 to 200, preferably from 0 to 10, more preferably from 0 to 7 and in particular 0, 1, 2, 3, 4, 5 or 6, and n stands for a number from 0 to 10, preferably from 1 to 8 and in particular for 2, 3, 4, 5, 6.

As additional silicones, water-soluble silicones may be present in the compositions according to the invention in the addition to the dimethicones, dimethiconols, amodimethicones and/or cyclomethicones according to the invention.

Corresponding hydrophilic silicones are selected from compounds of the formulas (Si-6) and/or (Si-7), for example. Particular preferred water-soluble silicone-based surfactants are selected from the group of dimethicone copolymers, which are preferably alkoxylated, in particular polyethoxylated or polypropoxylated.

The dimethicone copolymers preferred according to this invention are polyoxyalkylene-modified dimethylpolysiloxanes of the general formulas (Si-6) or (Si-7):

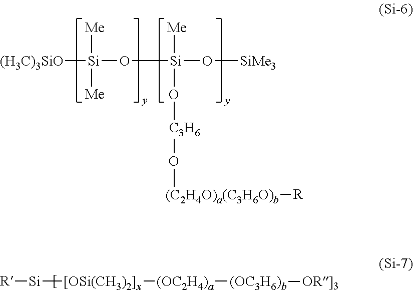

in which the radical R stands for a hydrogen atom, an alkyl group with 1 to 12 carbon atoms, an alkoxy group with 1 to 12 carbon atoms or a hydroxyl group, the radicals R′ and R″ denote alkyl groups with 1 to 12 carbon atoms, x stands for an integer from 1 to 100, preferably from 20 to 30, y stands for an integer from 1 to 20 preferably from 2 to 10 and a and b stand for integers from 0 to 50 preferably from 10 to 30.

Especially preferred dimethicone copolyols in the sense of this invention include, for example, the products distributed commercially under the brand name Silwet (Union Carbide Corporation) and Dow Corning. Especially preferred dimethicone copolyols according to the invention are Dow Corning 190 and Dow Corning 193.

The dimethicone copolyols are present in the compositions according to the invention in amounts of 0.01 to 10 wt %, preferably 0.01 to 8 wt %, especially preferably 0.1 to 7.5 wt % and in particular 0.1 to 5 wt % dimethicone copolyol, based on the composition.

Finally silicone compounds are understood to include the dimethiconols (Si-8). The dimethiconols according to the invention may be either linear or branched or cyclic or cyclic and branched. Linear dimethiconols can be represented by the following structural formula (Si-8-I):

(SiOHR¹₂)—O—(SiR²₂—O—)ₓ—(SiOHR¹₂) (Si-8-I)

Branched dimethiconols can be represented by the structural formula (Si-8-II)

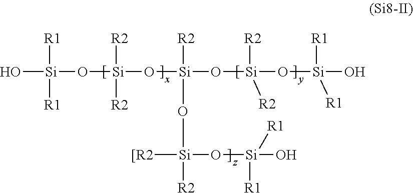

The radical R1 and R2, independently of one another, each stand for hydrogen, a methyl radical, a C₂ to C₃₀ linear, saturated or unsaturated hydrocarbon radical, a phenyl radical and/or an aryl radical. The numbers x, y and z are integers and amount to 1 to 50,000, independently of one another. The molecular weights of the dimethiconols are between 1000 D and 10,000,000 D. The viscosities are between 100 and 10,000,000 cPs measured at 25° C. with the help of a glass capillary viscometer according to Dow Corning Corporate test method CTM 0004 of Jul. 20, 1970. Preferred viscosities are between 1000 and 5,000,000 cPs, most especially preferred viscosities are between 10,000 and 3,000,000 cPs. The most preferred range is between 50,000 and 2,000,000 cPs.

The following commercial products can be mentioned as examples of such products: Dow Corning 1-1254 fluid, Dow Corning 2-9023 fluid, Dow Corning 2 9026 fluid, Abil OSW 5 (Degussa Care Specialties), Dow Corning 1401 fluid, Dow Corning 1403 fluid, Dow Corning 1501 fluid, Dow Corning 1784 HVF emulsion, Dow Corning 9546 silicone elastomer blend, SM555, SM2725, SM2765, SM2785 (all four aforementioned products are from GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforementioned products are from WackerChemie GmbH).

The methiconols (Si-8) are present in the compositions according to the invention in amounts of 0.01 to 10 wt %, preferably 0.01 to 8 wt %, especially preferably 0.1 to 7.5 wt % and in particular 0.1 to 5 wt % dimethiconol, based on the composition.

In addition, polymers are contained in an extremely preferred embodiment in order to increase the effect of the active ingredient complex (A).

The cationic polymers may be homopolymers or copolymers, wherein the quaternary nitrogen groups are present either in the polymer chain or preferably as a substituent on one or more of the monomers. The monomers containing ammonium groups cannot be copolymerized with noncationic monomers. Suitable cationic monomers include unsaturated radically polymerizable compounds which have at least one cationic group, in particular ammonium-substituted vinyl monomers, for example, trialkylmethacryloxyalkylammonium, trialkylacryloxyalkylammonium, dialkyldiallylammonium and quaternary vinyl ammonium monomers with cyclic, cationic groups containing nitrogen such as pyridinium, imidazolium or quaternary pyrrolidones, e.g., alkyl vinyl imidazolium, alkyl vinyl pyridinium or alkyl vinyl pyrrolidone salts. The alkyl groups of these monomers are preferably low alkyl groups, for example, C₁ to C₇ alkyl groups, especially preferably C₁ to C₃ alkyl groups.

The monomers containing ammonium groups may be copolymerized with noncationic monomers. Suitable comonomers include, for example, acrylamide, methacrylamide, alkyl and dialkylacrylamide, alkyl and dialkylmethacrylamide, alkyl acrylate, alkyl methacrylate, vinyl caprolactone, vinyl caprolactam, vinylpyrrolidone, vinyl esters, such as vinyl acetate, vinyl alcohol, propylene glycol or ethylene glycol wherein the alkyl groups of these monomers are preferably C₁ to C₇ alkyl groups, especially preferably C₁ to C₃ alkyl groups.

Suitable polymers with quaternary amine groups include, for example, the polymers described under the designations polyquaternium in the CTFA Cosmetic Ingredient Dictionary such as methyl vinyl imidazolium chloride/vinylpyrrolidone copolymer (polyquaternium-16) or quaternized vinylpyrrolidone/dimethylaminoethyl methacrylate copolymer (polyquaternium-11).

Of the cationic polymers that may be present in the agents according to the invention, vinylpyrrolidone/dimethylaminoethyl methacrylate methosulfate copolymer, for example, which is distributed by the company Gaf Co., USA under the brand names Gafquat® 755 N and Gafquat® 734 and of which Gafquat® 734 is especially preferred are suitable. Additional cationic polymers include, for example, the copolymer of polyvinylpyrrolidone and imidazolimine methochloride which is distributed by the company BASF Germany under the brand name Luviquat® HM 550, the terpolymer of dimethyldiallylammonium chloride, sodium acrylate and acrylamide distributed by the company Calgon USA under the brand name Merquat® Plus 3300 and the vinylpyrrolidone/methacrylamidopropyltrimethylammonium chloride copolymer distributed by the company ISP under the brand name Gafquat® HS 100.

Homopolymers of the general formula (P1),

{CH₂—[CR¹COO—(CH₂)ₘN⁺R²R³R⁴]}ₙX⁻

in which R¹═—H or —CH₃, R², R³ and R⁴, independently of one another, are selected from C₁₋₄ alkyl, alkenyl or hydroxyalkyl groups, m=1, 2, 3, or 4, n is a natural number and X⁻ is a physiologically tolerable organic or inorganic anion. Within the scope of these polymers, those that are preferred according to the invention are those for which at least one of the following conditions applies: R¹ stands for a methyl group, R², R³ and R⁴ stand for methyl groups, m has the value 2.

Examples of physiologically tolerable counterions X— include halide ions, sulfate ions, phosphate ions, methosulfate ions as well as organic ions such as lactate, citrate, tartrate and acetate ions. Halide ions are preferred, in particular chloride.

A particularly suitable homopolymer is poly(methacryloyloxyethyl-trimethylammonium chloride), optionally crosslinked, with the INCI designation polyquaternium-37. Such products are available commercially, for example, under the brand names Rheocare® CTH (Cosmetic Rheologies) and Synthalen® CR (3V Sigma).

The homopolymer is preferably used in the form of a nonaqueous polymer dispersion. Such polymer dispersions are commercially available under the brand names Salcare® SC 95 and Salcare® SC 96.

A copolymer that is preferred according to the invention is the crosslinked acrylamide methacryloyloxyethyltrimethyammonium chloride copolymer. Such copolymers are commercially available under the brand name Salcare® SC 92.

Suitable cationic polymers derived from natural polymers include cationic derivatives of polysaccharides, for example, cationic derivatives of cellulose, starch or guar. Also suitable are chitosan and chitosan derivatives.

Cationic polysaccharides have the general formula (P-3)

G-O—B—N+RₐR_{b}R_{c}X⁻,

[wherein] G is an anhydroglucose radical, for example, starch or cellulose anhydroglucose;
B is a divalent compound group, for example, alkylene, oxyalkylene, polyoxyalkylene or hydroxyalkylene;
Rₐ, R_{b} and R_{c} are, independently of one another, alkyl, aryl, alkylaryl, arylalkyl, alkoxyalkyl or alkoxyaryl, each with up to 18 carbon atoms, where the total number of carbon atoms in Rₐ, R_{b} and R_{c} is preferably max. 20;
X⁻ is a conventional counter-anion and is preferably chloride.

A cationic cellulose is distributed by Amerchol under the brand name Polymer JR® 400 and has the INCI designation polyquaternium-10. Another cationic cellulose has the INCI designation polyquaternium-24 and is distributed by Amerchol under the brand name LM-200. Additional commercial products include the compounds Celquat® H 100, Celquat® and L 200. The aforementioned commercial products are preferred cationic celluloses.

Suitable cationic guar derivatives are distributed under the commercial designation Jaguar® and have the INCI designation guar hydroxypropyltrimonium chloride. In addition, especially suitable cationic guar derivatives are also available commercially under the brand name N-Hance® from the Hercules company. Additional cationic guar derivatives are distributed by the Cognis company under the brand name Cosmedia®. A preferred cationic guar derivative is the commercial product AquaCat® from the Hercules company. This raw material is a predissolved cationic guar derivative.

A suitable chitosan is distributed, for example, by the Kyowa Oil & Fat company, Japan under the brand name Flonac®. A preferred chitosan salt is chitosonium pyrrolidone carboxylate which is distributed, for example, under the brand name Kytamer® PC by the Amerchol company, USA. Additional chitosan derivatives are available commercially under the brand names Hydagen® CMF, Hydagen® HCMF and Chitolam® NB/101.

Other preferred cationic polymers include, for example: cationic alkyl polyglycosides,

cationized honey, for example, the commercial product Honeyquat® 50,
polymeric dimethyldiallylammonium salts and their copolymers with esters and amides of acrylic acid and methacrylic acid. The products commercially available under the brand names Merquat® 100 (poly(dimethyldiallylammonium chloride)) and Merquat® 550 (dimethyldiallylammonium chloride-acrylamide copolymer) are examples of such cationic polymers
vinylpyrrolidone-vinyl imidazolium methochloride copolymers such as those available under the brand names Luviquat® FC 370, FC 550, FC 905 and HM 552, quaternized polyvinyl alcohol,
as well as the polymers with quaternary nitrogen atoms in the main polymer chain known by the designations polyquaternium 2, polyquaternium 17, polyquaternium 18 and polyquaternium 27,
vinylpyrrolidone-vinyl caprolactam-acrylate terpolymers such as those with acrylic acid esters and acrylic acid amides as the third monomer building block available commercially under the brand name Aquaflex® SF 40.

The copolymers of vinylpyrrolidone such as those obtainable as the commercial products Copolymer 845 (manufacturer ISP), Gaffix® VC 713 (manufacturer ISP), Gafquat® ASCP 1011, Gafquat® HS 110, Luviquat® 8155 and Luviquat® MS 370 can also be used according to the invention.

In addition, cationized protein hydrolyzates may also be included with the cationic polymers, wherein the underlying protein hydrolyzate may originate from animals, for example, from collagen, milk or keratin, from plants, for example, from wheat, corn, rice, potatoes, soy or almonds, from marine life forms, for example, from fish collagen or algae or biotechnologically produced protein hydrolyzates. Typical examples of the cationic protein hydrolyzates and derivatives according to the invention include the products covered by the INCI designations in the “International Cosmetic Ingredient Dictionary and Handbook,” (seventh edition 1997, The Cosmetic, Toiletry and Fragrance Association, 1101 17th Street, N.W., Suite 300, Washington, D.C. 20036-4702) and available commercially.

The cationic polymers are preferably contained in the compositions according to the invention in amounts of 0.01 to 10 wt %, based on the total agent. Amounts of 0.05 to 5 wt % are especially preferred.

In addition, amphoteric polymers may also be used as the polymers. The term amphoteric polymers is understood to include polymers having both free amino groups and free COOH or SO₃H groups in the molecule and capable of forming internal salts as well as zwitterionic polymers which contain quaternary ammonium groups and COO⁻ or SO₃⁻ groups in the molecule as well as those polymers which contain COOH or SO₃H groups and quaternary ammonium groups.

Amphoteric and/or cationic polymers preferred according to the invention include those polymers in which a cationic group is derived from at least one of the following monomers:

(i) monomers with quaternary ammonium groups of the general formula (Mono1)

R¹—CH═CR²—CO—Z—(CₙH₂ₙ)—N⁺R²R³R⁴A⁽⁻⁾ (Mono1)

in which R¹ and R², independently of one another, stand for hydrogen or a methyl group and R³, R⁴ and R⁵, independently of one another, stand for alkyl groups with 1 to 4 carbon atoms, Z is an NH group or an oxygen atom, N is an integer from 2 to 5 and A⁽⁻⁾ is the anion of an organic or inorganic acid,
(ii) Monomers with quaternary ammonium groups of the general formula (Mono2)

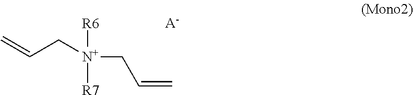

wherein R⁶ and R⁷, independently of one another, stand for a C₁ to C₄ alkyl group, in particular for a methyl group, and
A⁻ is the anion of an organic or inorganic acid,
(iii) monomeric carboxylic acids of the general formula (Mono3)

R⁸—CH═CR⁹—COOH (Mono3)

wherein R⁸ and R⁹, independently of one another, are hydrogen or methyl groups.

Especially preferred are those polymers in which the monomers of type (i) are used in which R³, R⁴ and R⁵ are methyl groups, Z is an NH group and A⁽⁻⁾ is a halide, methoxysulfate or ethoxysulfate ion; acrylamidopropyltrimethylammonium chloride is an especially preferred monomer (i). Acrylic acid is preferably used as monomer (ii) for the aforementioned polymers.

Especially preferred amphoteric polymers are copolymers from at least one monomer (Mono1) and/or (Mono2) with the monomer (Mono3) in particular copolymers of the monomers (Mono2) and (Mono3). Most especially preferred amphoteric polymers for use according to the invention are copolymers of diallyldimethylammonium chloride and acrylic acid. These copolymers are distributed under the INCI designation polyquaternium-22 with the brand name Merquat® 280 (Nalco) for example.

In addition, the amphoteric polymers according to the invention may also contain a monomer (Mono4), in addition to a monomer (Mono1) or (Mono2) and a monomer (Mono3):

(iv) monomeric carboxylic acid amides of the general formula (Mono4)

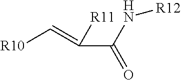

wherein R¹⁰ and R¹¹, independently of one another, are hydrogen or methyl groups and R¹² stands for a hydrogen atom or a C₁ to C₈ alkyl group.

Amphoteric polymers based on a copolymer (Mono4) that are most especially preferred for use according to the invention are the terpolymers of diallyldimethylammonium chloride, acrylamide and acrylic acid. These copolymers are distributed under the INCI designation polyquaternium-39 with the brand name Merquat® Plus 3330 (Nalco), among others.

The amphoteric polymers may be used in general according to the invention either directly or in salt form, which is obtained by neutralization of the polymers, for example, with an alkali hydroxide.

The amphoteric polymers are preferably present in the agents according to the invention in amounts of 0.05 to 10 wt %, based on the total agent. Amounts of 0.01 to 5 wt % are especially preferred.

The anionic polymers are anionic polymers which have carboxylate groups and/or sulfonate groups. Examples of anionic monomers of which such polymers may consist include acrylic acid, methacrylic acid, crotonic acid, maleic anhydride and 2-acrylamido-2-methylpropane sulfonic acid. The acidic groups may be present entirely or in part as the sodium, potassium, ammonium, mono- or triethanolammonium salts. Preferred monomers are 2 acrylamido-2-methylpropane sulfonic acid and acrylic acid.

Anionic polymers containing 2-acrylamido-2-methylpropane sulfonic acid as the sole monomer or as a comonomer have proven to be especially effective, where the sulfonic acid group may be present entirely or partially as the sodium, potassium, ammonium, mono- or triethanolammonium salt.

The homopolymer of 2-acrylamido-2-methylpropanesulfonic acid which is commercially available under the brand name Rheothik® 11-80, for example, is especially preferred.

Within this embodiment, it may be preferable to use copolymers of at least one anionic monomer and at least nonionic monomer. With respect to the anionic monomers, reference is made to the substances listed above. Preferred nonionic monomers include acrylamide, methacrylamide, acrylic acid esters, methacrylic acid esters, vinylpyrrolidone, vinyl ethers and vinyl esters.

Preferred anionic copolymers are acrylic acid acrylamide copolymers as well as in particular polyacrylamide copolymers with monomers that contain sulfonic acid groups. Such a polymer is contained in the commercial product Sepigel® 305 from the company SEPPIC.

The sodium acryloyldimethyl taurate copolymers distributed under the brand name Simulgel® 600 as a compound with isohexadecane and polysorbate 80 have also proven to be especially effective according to the invention.

Likewise preferred anionic homopolymers are crosslinked and uncrosslinked polyacrylic acid. Allyl ethers of pentaerythritol, sucrose and propylene may be preferred crosslinking agents. Such compounds are available commercially under the brand name Carbopol® for example.

Copolymers of maleic anhydride and methyl vinyl ether, in particular those with crosslinking, are also color preserving polymers. A maleic acid methyl vinyl ether copolymer crosslinked with 1,9-decadienes is available commercially under the brand name Stabileze® QM.

The anionic polymers are preferably used in the agents according to the invention in amounts of 0.05 to 10 wt %, based on the total agent. Amounts of 0.1 to 5 wt % are especially preferred.

A polyurethane that is most especially preferred according to the invention is commercially available under the brand name Luviset® PUR (BASF).

The agents according to the invention may contain nonionic polymers in another embodiment.

Suitable nonionic polymers include for example:

Vinylpyrrolidone/vinyl ester copolymers such as those distributed under the brand name Luviskol® (BASF). Luviskol® VA 64 and Luviskol® VA 73, both being vinylpyrrolidone vinyl acetate copolymers, are also preferred nonionic polymers.
Cellulose ethers such as hydroxypropyl cellulose, hydroxyethyl cellulose and methylhydroxypropyl cellulose such as those distributed, for example, under the brand names Culminal® and Benecel® (AQUALON) and Natrosol® products (Hercules)
Starch and derivatives thereof in particular starch ethers, for example, Structure® XL (National Starch), a multifunctional salt-tolerant starch.

### Shellac

Polyvinylpyrrolidones such as those distributed, for example, under the brand name Luviskol® (BASF).

The nonionic polymers are preferably present in the compositions according to the invention in amounts of 0.05 to 10 wt %, based on the total agent. Amounts of 0.1 to 5 wt % are especially preferred.

It is also possible according to the invention for the preparations used to contain several polymers, in particular two different polymers of the same charge and/or an anionic polymer and an amphoteric polymer and/or a nonionic polymer.

The polymers (P) are preferably contained in the compositions used according to the invention in amounts of 0.01 to 30 wt %, based on the total composition. Amounts of 0.01 to 25, in particular 0.01 to 15 wt % are especially preferred.

The compositions according to the invention may of course also contain the ingredients customarily used in cosmetic compositions.

Emulsifiers that may be used according to the invention include for example
addition products of 4 to 30 mol ethylene oxide and/or 0 to 5 mol propylene oxide onto linear fatty alcohols with 8 to 22 carbon atoms, onto fatty acids with 12 to 22 carbon atoms and onto alkyl phenols with 8 to 15 carbon atoms in the alkyl group,
C₁₂-C₂₂ fatty acid mono- and diesters of addition products of 1 to 30 mol ethylene oxide onto polyols with 3 to 6 carbon atoms in particular onto glycerol
ethylene oxide and polyglycerol addition products onto methyl glucoside fatty acid esters, fatty acid alkanolamides and fatty acid glucamides,
C₈-C₂₂ alkylmono- and oligoglycoside and their ethoxylated analogs, where degree of oligomerization of 1.1 to 5, in particular 1.2 to 2.0 and glucose as the sugar component are preferred,
mixture of alkyl (oligo)glucosides and fatty alcohols, for example, the commercially available product Montanov® 68,
addition products of 5 to 60 mol ethylene oxide onto castor oil and hydrogenated castor oil,
partial esters of polyols with 3 to 6 carbon atoms with saturated fatty acids with 8 to 22 carbon atoms,
sterols both from animal tissue (zoosterols, cholesterols, lanosterol) and from vegetable fats (phytosterols, ergosterol, stigmasterol, sitosterol) or from fungi and yeast (mycosterols),
phospholipids (lecithins, phosphatidylcholines),
fatty acid esters of sugars and sugar alcohols such as sorbitol,
polyglycerols and polyglycerol derivatives, for example, polyglycerol poly-12-hydroxystearate (commercial product Dehymuls® PGPH).

The agents according to the invention preferably contain the emulsifiers in amounts of 0.1-25 wt %, in particular 0.5-15 wt %, based on the total agent.

The fatty acids (Fatac) used may be linear and/or branched, saturated and/or unsaturated fatty acids with 6 to 30 carbon atoms. Fatty acids with 10 to 22 carbon atoms are preferred. These include, for example, the isostearic acids such as the commercial product Emersol® 871 and Emersol® 875 and isopalmitic acids such as the commercial product Edenor® IP 95 as well as all other fatty acids distributed under the brand names Edenor® (Cognis). Other typical examples of such fatty acids include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucaic acid as well as their technical-grade mixtures. Especially preferred are usually the fatty acid cuts which can be obtained from coconut oil or palm oil; use of stearic acid is usually preferred.

The quantity used is 0.1-15 wt %, based on the total agent. In a preferred embodiment the quantity is 0.5-10 wt %, but quantities of 1-5 wt % are most especially advantageous.

Saturated, mono- or polyunsaturated, branched or unbranched fatty alcohols (Fatal) with C₆-C₃₀, preferably C₁₀-C₂₂ and most especially C₁₂-C₂₂ carbon atoms may be used as the fatty alcohols. In the sense of the invention, decanol, octanol, octenol, dodecenol, decenol, octadienol, dodecadienol, decadienol, oleyl alcohol, erucaic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol as well as their Guerbet alcohols may be used in the sense of this invention, but this list is intended only as an example and should not restrict the invention in any way. However, the fatty alcohols are derived from preferably naturally fatty acids, and it may usually be assumed that they are produced from the esters of the fatty acids by reduction. Likewise fatty alcohol cuts which are a mixture of different fatty alcohols may also be used according to the invention. Such substances are available commercially, for example, under the brand names Stenol®, e.g., Stenol® 1618 or Lanette®, e.g., Lanette® 0 or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Lanolin alcohols such as those available commercially under the brand names Corona®, White Swan®, Coronet® or Fluilan® may of course also be used according to the invention. The fatty alcohols are used in amounts of 0.1-30 wt %, based on the total preparation, preferably in amounts of 0.1-20 wt %.

According to the invention, solid paraffins or isoparaffins, carnauba waxes, beeswax, candelilla waxes, ozokerites, ceresins, sperm oil, sunflower wax, fruit waxes such as apple wax or citrus wax, microwaxes of PE or PP may also be used as natural or synthetic waxes (fat wax) according to the invention. Such waxes are available commercially, for example, from the company Kahl & Co. Trittau.

The use quantity is 0.1-50 wt %, based on the total agent preferably 0.1-20 wt % and especially preferably 0.1-15 wt %, based on the total agent.

The total amount of oil and fat components in the agents according to the invention is usually 0.5-75 wt %, based on the total agent. Amounts of 0.5-35 wt % are preferred according to the invention.

Another synergistic active ingredient according to the invention in the compositions according to the invention, comprising the active ingredient complex according to the invention are protein hydrolyzates and/or derivatives thereof (P).

Protein hydrolyzates of both plant and animal or marine or synthetic origin may be used according to the invention.

Animal protein hydrolyzates include, for example, elastin, collagen, keratin, silk and milk protein hydrolyzates which may also be present in the form of salts. Such products are distributed, for example, under the brand names Dehylan® (Cognis), Promois® (Interorgana), Collapuron® (Cognis), Nutrilan® (Cognis), Gelita-Sol® (Deutsche Gelatine Fabriken Stoess & Co.), Lexein® (Inolex) and Kerasol® (Croda).

In addition, plant protein hydrolyzates that are especially preferred according to the invention include, for example, soy, almond, pea, moringa, potato and wheat protein hydrolyzates. Such products are available commercially, for example, under the brand names Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 available from the company Laboratoires Sérobiologiques.

Additional protein hydrolyzates preferred according to the invention are of maritime origin. These include, for example, collagen hydrolyzates from fish or algae and protein hydrolyzates from mussels and/or pearl hydrolyzates. Examples of pearl extracts according to the invention include the commercial products Pearl Protein Extract BG® or Crodarom® Pearl.

It is extremely preferred, however, if casein is used as the protein hydrolyzate. The use of casein in the compositions together with rambutane oil leads to definitely sensorially perceptible haptic effects on the hair and skin. Thus hair treated in this way feels much stronger due to its haptic properties. The surface is smooth and flexible on the one hand while on the other hand there is still a perceptible covering of the surface with the active ingredient combination.

The protein hydrolyzates (P) are present in the compositions in concentrations of 0.001 wt % to 20 wt %, preferably from 0.05 wt % to 15 wt % and most especially preferably in amounts of 0.05 wt % to 5 wt %.

The effect of the compositions according to the invention can be increased further by a 2-pyrrolidinone-5-carboxylic acid and its derivatives (J). The sodium, potassium, calcium, magnesium or ammonium salts in which the ammonium ion also has one to three C₁ to C₄ alkyl groups in addition to hydrogen are preferred. The sodium salt is most especially preferably preferred. The quantities used in the agents according to the invention preferably amount to 0.05 to 10 wt %, based on the total agent, especially preferably 0.1 to 5 and in particular 0.1 to 3 wt %.

Another preferred group of ingredients of the compositions according to the invention with the active ingredient complex according to the invention is vitamins, provitamins or vitamin precursors. Vitamins, provitamins or vitamin precursors that can be attributed to the groups A, B, C, E, F and H are especially preferred.

The group of substances referred to as vitamin A includes retinol (vitamin A1) and 3,4-didehydroretinol (vitamin A2). The β-carotine is the provitamin of retinol. For example, vitamin A acid and its esters, vitamin A aldehyde and vitamin A alcohol as well as its esters and the palmitate and acetate may be considered as the vitamin A component according to the invention. The agents according to the invention preferably contain the vitamin A component in amounts of 0.05-1 wt %, based on the total preparation.

Vitamin C (ascorbic acid). Vitamin C is preferably used in the agents according to the invention in amounts of 0.1 to 3 wt %, based on the total agent. Use in the form of palmitic acid ester, the glucosides or phosphates may be preferred. Use in combination with tocopherols may also be preferred.

Vitamin E (tocopherol in particular α-tocopherol). Tocopherol and its derivatives including in particular the esters such as the acetate, the nicotinate, the phosphate and the succinate, are preferably present in the agents according to the invention in amounts of 0.05-1 wt %, based on the total agent.

Vitamin F. The term “vitamin F” is usually understood to refer to essential fatty acids in particular linoleic acid, linolenic acid and arachidonic acid.

Vitamin H. The compound (3aS,4S,6aR)-2-oxohexahydrothienol[3,4-d]-imidazole-4-valeric acid is also referred to as vitamin H but in the meantime the trivial name biotin has become popular for this substance. Biotin is preferably contained in the agents according to the invention in amounts of 0.0001 to 1.0 wt %, in particular in amounts of 0.001 to 0.01 wt %.

The compositions according to the invention preferably contain vitamins, provitamins and vitamin precursors from groups A, E and H.

An especially preferred group of ingredients in the cosmetic compositions according to the invention are the following betaines: carnitine, carnitine tartrate, carnitine magnesium citrate, acetyl carnitine, betalaine, 1,1-dimethylproline, choline, choline chloride, choline bitartrate, choline dihydrogen citrate and the compound N,N,N-trimethylglycine referred to in the literature as betaine.

The preferred betaines for use are carnitine, histidine, choline and betaine. In an especially preferred embodiment of the invention, L-carnitine tartrate is used as an active ingredient.

Taurine and/or a derivative of taurine is an especially important ingredient. Taurine is understood exclusively to be 2-aminoethanesulfonic acid and a derivative of taurine is understood to be those derivatives of taurine mentioned explicitly. The derivatives of taurine are understood to be in monomethyltaurine, N,N-dimethyltaurine, taurine lysylate, taurine tartrate, taurine ornithate, lysyl taurine and ornithyl taurine. Further taurines in the sense of the present invention are taurocholic acid and hypotaurine.

Agents according to the invention containing—based on their weight—0.0001 to 10.0 wt %, preferably 0.0005 to 5.0 wt %, especially preferably 0.001 to 2.0 wt % and in particular 0.001 to 1.0 wt % taurine and/or a derivative of taurine are especially preferred.

In another preferred embodiment according to the invention, the compositions according to the invention contain bioquinones. Suitable bioquinones in the agents according to the invention are understood to be one or more ubiquinones and/or plastoquinones. The preferred ubiquinones according to the invention have the following formula:

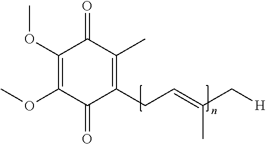

where n=6, 7, 8, 9 or 10.

Coenzyme Q10 is most preferred here.

Preferred compositions according to the invention contain purine and/or purine derivatives in narrow quantity ranges. Preferred cosmetic agents according to the invention are characterized in that they contain—based on their weight—0.001 to 2.5 wt % preferably 0.0025 to 1 wt %, especially preferably 0.005 to 0.5 wt % and in particular 0.01 to 0.1 wt % purine(s) and/or purine derivative(s). Preferred cosmetic agents according to the invention are characterized in that they contain purine, adenine, guanine, uric acid, hypoxanthine, 6-purinethiol, 6 thioguanine, xanthine, caffeine, theobromine or theophylline. Caffeine is the most preferred in hair cosmetic preparation.

In another preferred embodiment of the present invention the cosmetic agent contains ectoine ((S)-2-methyl-1,4,5,6-tetrahydro-4-pyrimidine carboxylic acid).

Especially preferred according to the invention are agents which contain—based on their weight—0.00001 to 10.0 wt % preferably 0.0001 to 5.0 wt % and in particular 0.001 to 3 wt % of active ingredients from the group formed by carnitine tartrate, taurine, coenzyme Q10, ectoine, a purine, in particular caffeine and derivatives thereof or physiologically acceptable salts.

In another embodiment, the agents according to the invention should additionally contain at least one UV light protective filter. UVB filters may be oil-soluble or water-soluble.

Examples of oil-soluble substances that can be mentioned include:
3-benzylidene camphor, e.g., 3-(4-methylbenzylidene camphor),
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester;
esters of cinnamic acid, preferably 4-methoxycinnamic acid 2-ethylhexyl ester, 4-methoxycinnamic acid propyl ester, 4-methoxycinnamic acid isoamyl ester, 2-cyano-3-phenylcinnamic acid 2-ethylhexyl ester (octocrylene);
esters of salicylic acid, preferably salicylic acid 2-ethylhexyl ester, salicylic acid 4-isopropylbenzyl ester, salicylic acid homomethyl ester;
derivatives of benzophenone, preferably 2-hydroxy-4-methoxybenzophenone, 2-hydroxy-4-methoxy-4′-methylbenzophenone, 2,2′-dihydroxy-methoxybenzophenone;
esters of benzalmalonic acid, preferably 4-methoxybenzmalonic acid di-2-ethylhexyl ester;
triazine derivatives, for example, 2,4,6-trianilino-(p-carbo-2′-ethyl-1′-hexyloxy)-1,3,5-triazine and octyltriazone;
propane-1,3-diones, for example, 1-(4-tert-butylphenyl)-3-(4′-methoxy-phenyl)propane-1,3-dione.

Water-soluble substances that may be used include
2-phenylbenzimidazole-5-sulfonic acid and the alkali, alkaline earth, ammonium, alkylammonium, allcanolammonium and glucammonium salts thereof;
sulfonic acid derivatives of benzophenones preferably 2-hydroxy-4-methoxy-benzophenone-5-sulfonic acid and salts thereof;
sulfonic acid derivatives of 3-benzylidene camphor, for example, 4-(2-oxo-3-bornylidenemethyl)benzenesulfonic acid and 2-methyl-5-(2-oxo-3-bornylidene)sulfonic acid and salts thereof.

Typical UVA filters include in particular derivatives of benzoylmethane, for example, 1-(4′-tert-butylphenyl)-3-(4′-methoxyphenyl)propane-1,3-dione or 1 phenyl-3-(4′-isopropylphenyl)propane-1,3-dione. The UVA and UVB filter may of course also be used in mixtures. In addition to the aforementioned soluble substances, insoluble pigments in particular finely dispersed metal oxides and/or salts may also be used for this purpose, for example, titanium dioxide, zinc oxide, iron oxide, aluminum oxide, cerium oxide, zirconium oxide, silicate (talc), barium sulfate and zinc stearate. The particles should have an average diameter of less than 100 nm, preferably between 5 and 50 nm and in particular between 15 and 30 mm. They may have a spherical shape but particles having an ellipsoidal shape or some other shape deviating from the spherical may also be used.

Finally, additional advantages are achieved through the use of plant extracts (L) in the compositions according to the invention. According to the invention, in particular the extracts from green tea, oak bark, stinging nettle, witch hazel, hops, henna, chamomile, burdock root, horsetail, hawthorne, lime tree blossoms, almond, aloe vera, pine needle, horse chestnut, sandalwood, juniper berry, coconut, mango, apricot, lime, wheat, kiwi, melon, orange, grapefruit, sage, rosemary, birch, mallow, lady's smock, wild thyme, yarrow, thyme, melissa, rest harrow, coltsfoot, marsh mallow, meristem, ginseng, coffee, cocoa, moringa, ginger root and ayurvedic plant extracts, for example, Aegle marmelos (bilwa), Cyperus rotundus (nagar motha), Emblica officinalis (amalki), Morida citrifolia (ashyuka), Tinospora cordifolia (guduchi), Santalum album (chandana), Crocus satifus (kumkuma), Cinnamonum zeylanicum and Nelumbo nucifera (kamala, sacred lotus), sweet grasses such as wheat, barley, rye, oats, spelt, corn, the different varieties of sorghum (for example, proso millet, crabgrass, foxtail millet), sugar cane, rye grass, meadow foxtail, oatgrass, pasture grass, meadow fescue, purple moor grass, bamboo, cotton grass fountain grasses and foxtail grasses, andropogonodeae (Imperata cylindrica, sword grass, also known as flame grass or cogon grass), buffalo grass, cord grasses, dogtooth grasses, love grasses, cymbopogon (lemongrass), oryzeae (rice), zinnia (wild rice), beach grass, blue oat grasses, honey grasses, quaking grasses, meadow grasses, couchgrass and echinacea, in particular Echinacea angustifolia DC, Echinacea paradoxa (Norton), Echinacea simulate, E. atrorubens, E. tennesiensis, Echinacea strigosa (McGregor), Echinacea laevigata, Echinacea purpurea (L.) Moench and Echinacea pallida (Nutt), all types of wine and pericarp of Litchie chinensis.

The plant extracts may be used according to the invention either in pure form or in dilute form. If they are used in dilute form, the usually contain approx. 2-80 wt % active substance and as the solvent the extraction agent or extraction agent mixture used to extract them.

Furthermore the cosmetic agents may contain additional active ingredients, excipients and additives, such as for example:
structurants such as maleic acid and lactic acid
swelling agents such as urea, allantoin, carbonates or hydantoin
dimethylisosorbide and cyclodextrins
dyes for coloring the agent,
antidandruff active ingredients such as piroctone olamine, zinc omadine and climbazol,
chelating agents such as EDTA, NTA, β-alanine diacetic acid and phosphonic acids,
opacifiers such as latex, styrene/PVP and styrene/acrylamide copolymers
pearlizing agents such as ethylene glycol mono- and distearate as well as PEG-3 distearate
pigments
stabilizers for hydrogen peroxide and other oxidizing agents
propellants such as propane-butane mixtures, N₂O, dimethyl ether, CO₂ and air
antioxidants
perfume oils, scents and fragrances.

With respect to additional optional components as well as the quantities of these components used, reference is made explicitly to the relevant textbooks with which the skilled person is familiar.

An additional subject matter of the invention is a method for treating keratinic fibers in which a cosmetic agent according to claim 1 is applied to the keratinic fibers in particular human hair and is rinsed out again after a treatment time of the keratinic fibers.

The treatment time is preferably a few seconds to 100 minutes, especially preferably 1 to 50 minutes and most especially preferably 1 to 30 minutes.

Another subject matter of the present invention is the use of the composition according to the invention to improve the elasticity of keratinic fibers.

Another subject matter of the present invention is the use of the composition according to the invention to improve the elasticity of keratinic fibers.

Another subject matter of the present invention is the use of the composition according to the invention to improve the combability of the keratinic fibers.

### EXAMPLES

All quantitative information is parts by weight unless otherwise indicated. The following formulations have been prepared using known preparation methods

Hair Rinse

| | S1 | S2 | S3 | S4 | S5 | S6 | S7 | S8 |
|---|---|---|---|---|---|---|---|---|
| Stenol ® 1618 | 3.0 | 3.0 | 3.0 | 3.0 | 3.0 | 3.0 | 3.0 | 3.0 |
| Tegoamid ® | 1.0 | 1.0 | 1.0 | 1.0 | 1.0 | 1.0 | 1.0 | 1.0 |
| S 18 | | | | | | | | |
| Rheocare ® | 2.0 | 2.0 | 2.0 | 2.0 | 2.0 | 2.0 | 2.0 | 2.0 |
| Ultragel | | | | | | | | |
| Panthenol | — | 0.2 | — | 0.2 | — | 0.2 | — | 0.2 |
| Mirustyle ® | 2.0 | 2.0 | 2.0 | 2.0 | 2.0 | 2.0 | 2.0 | 2.0 |
| CP (contains | | | | | | | | |
| up to 25 wt % | | | | | | | | |
| polyquatenium- | | | | | | | | |
| 72, remainder | | | | | | | | |
| water) | | | | | | | | |
| Dow | — | — | 0.5 | 0.5 | — | — | 0.5 | 0.5 |
| Corning ® | | | | | | | | |
| DC 959 | | | | | | | | |
| Quaternium-91 | — | — | — | — | 1.0 | 1.0 | 1.0 | 1.9 |
| Methylparaben | 0.2 | 0.2 | 0.2 | 0.2 | 0.2 | 0.2 | 0.2 | 0.2 |
| Perfume | 0.3 | 0.3 | 0.3 | 0.3 | 0.3 | 0.3 | 0.3 | 0.3 |
| Phenoxy- | 0.4 | 0.4 | 0.4 | 0.4 | 0.4 | 0.4 | 0.4 | 0.4 |
| ethanol | | | | | | | | |
| Water | to | to | to | to | to | to | to | to |
| | 100 | 100 | 100 | 100 | 100 | 100 | 100 | 100 |

The pH values of all the formulations were adjusted to 2 to 4. Instead of quaternium-91, Alcypoquat® 131, Incroquat® Behenyl HE, Armocare® VGH-70, Luviquat® CP or Dehyquart® L80 may be used in the same amount with respect to the cationic active substance and achieve the same effect.

Deep Conditioner for Hair

| | K1 | K2 | K3 | K4 | K5 | K6 | K7 | K8 |
|---|---|---|---|---|---|---|---|---|
| Stenol ® 1618 | 6.0 | 6.0 | 6.0 | 6.0 | 6.0 | 6.0 | 6.0 | 6.0 |
| Tegoamid ® | 1.5 | 1.5 | 1.5 | 1.5 | 1.5 | 1.5 | 1.5 | 1.5 |
| S 18 | | | | | | | | |
| Rheocare ® | 3.0 | 3.0 | 3.0 | 3.0 | 3.0 | 3.0 | 3.0 | 3.0 |
| Ultragel | | | | | | | | |
| Panthenol | — | 0.5 | — | 0.5 | — | 0.5 | — | 0.5 |
| Mirustyle ® | 3.0 | 3.0 | 3.0 | 3.0 | 3.0 | 3.0 | 3.0 | 3.0 |
| CP (contains | | | | | | | | |
| up to 25 wt % | | | | | | | | |
| polyquatenium- | | | | | | | | |
| 72, remainder | | | | | | | | |
| water) | | | | | | | | |
| Dow | — | — | 1.0 | 1.0 | — | — | 1.0 | 1.0 |
| Corning ® | | | | | | | | |
| DC 959 | | | | | | | | |
| Quaternium-91 | — | — | — | — | 1.5 | 1.5 | 1.5 | 1.5 |
| Methylparaben | 0.2 | 0.2 | 0.2 | 0.2 | 0.2 | 0.2 | 0.2 | 0.2 |
| Perfume | 0.3 | 0.3 | 0.3 | 0.3 | 0.3 | 0.3 | 0.3 | 0.3 |
| Phenoxy- | 0.4 | 0.4 | 0.4 | 0.4 | 0.4 | 0.4 | 0.4 | 0.4 |
| ethanol | | | | | | | | |
| Water | to | to | to | to | to | to | to | to |
| | 100 | 100 | 100 | 100 | 100 | 100 | 100 | 100 |

The pH values of all the formulations were adjusted to 2 to 4. Instead of quaternium-91, Akypoquat® 131, Incroquat® Behenyl HE, Armocare® VGH-70, Luviquat® CP or Dehyquart® L80 may be used in the same amount with respect to the cationic active substance and achieve the same effect.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair cosmetic agent for treatment of keratinic fibers, comprising:
a) at least 0.01 wt % polyquaternium-72;
b) at least 0.01 wt % of at least one amine; and
c) a cosmetic vehicle.

2. The hair cosmetic agent according to claim 1, wherein the at least one amine comprises a cationized amine.

3. The hair cosmetic agent according to claim 1, further comprising at least one vitamin of the B series.

4. The hair cosmetic agent according to claim 1, further comprising at least one silicone compound selected from the group consisting of dimethicone, dimethiconol, and amino-functional silicone.

5. The hair cosmetic agent according to claim 1, further comprising at least one compound selected from carnitine tartrate, taurine, coenzyme Q10, ectoine, a purine, in particular, caffeine, and their physiologically acceptable salts.

6. The hair cosmetic agent according to claim 1, wherein the at least one amine comprises an amidoamine.

7. The hair cosmetic agent according to claim 1, wherein the at least one amine comprises a cationized amidoamine.

8. A method for cosmetic treatment of keratinic fibers, comprising:
applying an agent according to claim 1 to the keratinic fibers; and
rinsing off the keratinic fibers after a treatment time of a few seconds up to 100 minutes.
